(19) United States
(12) Patent Application Publication
(10) Pub. No.: US 2017/0060256 A1
(43) Pub. Date: Mar. 2, 2017

(54) SYSTEM AND METHOD FOR CONTROLLING AN ELECTRONIC DEVICE WITH A FACIAL GESTURE CONTROLLER

(71) Applicants: Sandy Lawrence Heck, Los Angeles, CA (US); Michael Rollin, Denver, CO (US); Eric Conley, South Berwick, ME (US)

(72) Inventors: Sandy Lawrence Heck, Los Angeles, CA (US); Michael Rollin, Denver, CO (US); Eric Conley, South Berwick, ME (US)

(73) Assignee: REACH BIONICS, INC., LOS ANGELES, CA (US)

(21) Appl. No.: 15/252,399

(22) Filed: Aug. 31, 2016

Related U.S. Application Data
(60) Provisional application No. 62/212,523, filed on Aug. 31, 2015.

(51) Int. Cl. G06F 3/01; A61F 9/02

(52) U.S. Cl. CPC G06F 3/017; G06F 3/012; A61F 9/029; A61F 9/027

(57) ABSTRACT

A facial gesture controller for controlling an electronic device is provided. The facial gesture controller includes a body, a plurality of sensors, and at least one processor. The body is configured to fit over the face of a user. The plurality of sensors are disposed on the body. The at least one processor is in electrical communication with the sensors. The sensors generate and transmit electrical signals to the at least one processor in response to movements of one or more facial muscles of the user. The at least one processor generates and transmits control signals corresponding to the electrical signals, received from the sensors, to the electronic device.

### CROSS-REFERENCE TO RELATED APPLICATIONS

This Application claims priority to U.S. Provisional Application Ser. No. 62/212,523, filed on Aug. 31, 2015 and entitled “FACIAL GESTURE CONTROLLER”, which is incorporated herein by references in its entirety.

### BACKGROUND

Technical Field

Embodiments of the invention relate generally to user interfaces for controlling electronic devices, and more specifically, to a system and method for controlling an electronic device via a facial gesture controller.

Discussion Of Art

Measuring voltages generated by living tissue is important in certain clinical and biomedical applications. Voltages generated from muscles are typically measured in a clinical setting to diagnose neuromuscular disorders. While such voltages have been successfully tested as command signals for prosthetics and other biomedical applications, such applications typically require a user to wear cumbersome sensing equipment that must be carefully positioned.

Electromyography (“EMG”) is a diagnostic tool that measures the voltages generated from muscles, and is typically used to assess the health of muscular nerves. The human nerve system transmits small electrical signals that cause muscles to contract and expand. EMG captures these signals and translates them into a means that can be visualized and measured. Special electrodes are used to sense these small electromagnetic signals. In particular, EMG measures the electrical activity of muscles during rest, slight contraction and forceful contraction. As such, EMG is usually associated as being a function of time described in terms of strength (amplitude) and frequency.

Jan Swammerdam (1637-1680), a Dutch anatomist and biologist, was the first to discover that stroking the innervating nerve of a frog's gastrocnemius muscle, located on the back portion of the lower leg, caused the muscle to contract and move.

Francisco Redi (1626-1698) was the first to recognize the connection between muscles and the generation of electricity.

Years later, Allesandro Volta (1745-1827), an Italian physics and chemist, invented the first electric battery, which would be used by others to stimulate muscles.

Luigi Galvani (1737-1798), an Italian physician and physicist credited as being the father of Neurophysiology, discovered that the leg muscles of a dead frog twitched when struck by an electrical spark.

Inspired by the work of Luigi Galvani, Carlo Matteucci (1811-1868), a giant in his field, used a Galvanometer to show that nerves can carry electrical signals to the muscles causing muscular contractions. A galvanometer is an electromagnetic device that is capable of determining small electric currents and rendering them visible in the form of a gauge.

Guillaume Duchenne (1806-1875), a French neurologist, further continued Galcani's studies. Guillaume wanted to determine how the human face produced facial expressions and began experimenting with electrical probes, triggering muscular contractions. He systematically mapped out functions of nearly every facial muscle. He often used one of his patients, whom he referred to as the “Old Man” for having no teeth and lacking facial expressions, to demonstrate the various muscles that control facial expressions. By passing a current through a specific facial muscle, Guillaume could trigger specific facial expressions within the Old Man's face. As a result, Guillaume is credited with discovering, through the use of electricity, that smiles resulting from true happiness, generally, not only utilizes the muscles of the mouth, but also the muscles around the eyes. He further found that an insincere smile often involves only using the muscles of the mouth. This discovery is often referred to as the “Duchenne smile.”

Accordingly, there are typically eight basic emotions that humans express in their facial expressions which are commonly referred to as: Anger; Fear; Disgust; Contempt; Joy; Surprise; Sadness; and No Expression. In response to these emotional states, specific facial gestures take place, which use a reliable set of facial muscles that all activate at the same time and produce electromagnetic signals of particular patterns.

When expressing Anger, the eyebrows tend to pull down, the upper lids pull down, the lower lids pull up, and the margins between the lips are rolled in. When expressing Fear, the eyebrows are pulled up and together, the upper eyelids are pulled up, and the mouth is opened or stretched. When expressing Disgust, the eyebrows are pulled down, the nose is wrinkled, the upper lip is pulled up, and the lips are loose. When expressing Contempt, the eyes are neutral and there are unilateral lip expressions. When the facial expression of Joy is demonstrated, the muscles around the eyes tighten, the cheeks are raised, the lip corners are raised diagonally, and there is a wrinkle around the eyes. If there is a smile without the eye muscles contracting, it is typically understood that the smile is not sincere. When expressing Surprise, the entire eyebrow is pulled up, the eyelids are also pulled up, and the mouth hangs open. When Sadness is expressed, the inner corners of the eyes are raised, the eyelids are loose, and the lip corners are pulled down. Finally, in a Neutral position, e.g., No Expression, all the facial muscles are typically in the position of rest.

There are also other forms of idiosyncratic facial expressions that use uncommon sets of facial muscles, and which typically do not reflect one of the common eight emotions mentioned above. All of the aforementioned facial expressions, however, generate patterns of electrical signals across the face.

Generally, human communication takes on many forms. For example, there are verbal forms, e.g., speech recognition, and nonverbal forms, e.g., body language, voice tone, touch, body posture, head tilt, appearance, hand gestures, facial expressions, eye contact, and/or head nodding. In nonverbal communication, some forms of communication are deliberate while others are performed unknowingly. Examples of nonverbal communication include crossed legs, crossed arms, and/or demonstrating defiance or disagreement. Environment may also contribute to the meaning of nonverbal communication, e.g., having a meeting at a restaurant, or having the same meeting at a police station. Similarly, the position where one sits at a table may also send nonverbal communication, e.g., sitting at the head of the table vs. sitting on the sides of the table. For example, people arguing points tend to sit opposite each other, whereas people engaged in negotiations, when collaboration is desired, tend to sit side-by-side. The time a person gives another during conversations may also communicate nonverbally a showing of deference or respect. Touching is yet another form of nonverbal communication. For example, the firmness of a person's hand shake, e.g., good, firm, too strong, or weak, is a form of nonverbal communication.

As it happens to be, many human conversations take place with the assistance of electronic devices, e.g., phones, video-teleconferencing systems, and/or virtual reality based systems. However, unlike verbal communications, which can be typed, or recorded and transmitted, it is often difficult to convey nonverbal verbal communications over an electronic medium.

Additionally, many virtual reality (“VR”) systems require a user to wear a headset that restricts vision of their physical hands, thereby making hand-based controllers uncomfortable and uncertain to use. Accordingly, many such VR systems create a desire in the user to remove the headset, so as to look directly at their hands. Removing the headset, however, may take the user out of the VR experience—resulting in disorientation. Moreover, since audio is an important part of the VR experience, voice commands are not always compatible with game play or other VR uses. Further, motion tracking requires externally mounted or worn sensors that add unnecessary equipment and require the user to remain in a position that may be hard to maintain when they are immersed in the VR world.

Accordingly, there is a need for a method and system for measuring facial gestures and understanding their representations. More specifically, there is a need for a system and method for controlling an electronic device via a facial gesture controller.

### BRIEF DESCRIPTION

In an embodiment, a facial gesture controller for controlling an electronic device is provided. The facial gesture controller includes a body, a plurality of sensors, and at least one processor. The body is configured to fit over the face of a user. The plurality of sensors are disposed on the body. The at least one processor is in electrical communication with the sensors. The sensors generate and transmit electrical signals to the at least one processor in response to movements of one or more facial muscles of the user. The at least one processor generates and transmits control signals corresponding to the electrical signals, received from the sensors, to the electronic device.

In another embodiment, a computerized system is provided. The system includes an electronic device, and a facial gesture controller. The facial gesture controller includes at least one processor, a body configured to fit over the face of a user, and a plurality of sensors disposed on the body. The sensors generate and transmit electrical signals to the at least one processor in response to movements of one or more facial muscles of the user. The at least one processor generates and transmits control signals corresponding to the electrical signals, received from the sensors, to the electronic device.

In yet another embodiment, a method of controlling an electronic device is provided. The method includes: generating electrical signals in response to movements of one or more facial muscles of a user via a plurality of sensors disposed on a body of a facial gesture controller, the body configured to fit over the face of the user; transmitting the generated electrical signals to at least one processor; generating control signals based at least in part on the electrical signals via the at least one processor; and transmitting the control signals to the electronic device via the at least one processor.

### DRAWINGS

The present invention will be better understood from reading the following description of non-limiting embodiments, with reference to the attached drawings, wherein below:

FIG. 1 is a perspective view of a facial gesture controller for controlling an electronic device in accordance with an embodiment of the present invention;

FIG. 2 is another perspective view of the facial gesture controller of FIG. 1, wherein electrodes are attached to a first side of a body of the controller, in accordance with an embodiment of the present invention;

FIG. 3 is another perspective view of the facial gesture controller of FIG. 1 in accordance with an embodiment of the present invention;

FIG. 4 is another perspective view of the facial gesture controller of FIG. 1 in accordance with an embodiment of the present invention;

FIG. 5 is a block diagram of a system for controlling an electronic device that utilizes the facial gesture controller of FIG. 1 in accordance with an embodiment of the present invention;

FIG. 6 is another perspective view of the facial gesture controller of FIG. 1, wherein the facial gesture controller forms part of a pair of goggles, in accordance with an embodiment of the present invention;

FIG. 7 is another perspective view of the facial gesture controller of FIG. 1, wherein the facial gesture controller is configured for use independent of a goggle assembly, in accordance with an embodiment of the present invention;

FIG. 8 is another perspective view of the facial gesture controller of FIG. 1, wherein an at least one processor of the facial gesture controller is mounted to a strap connected to the facial gesture controller, in accordance with an embodiment of the present invention; and

FIG. 9 is cross-sectional view of a sensor of the facial gesture controller of FIG. 1 in accordance with an embodiment of the present invention.

### DETAILED DESCRIPTION

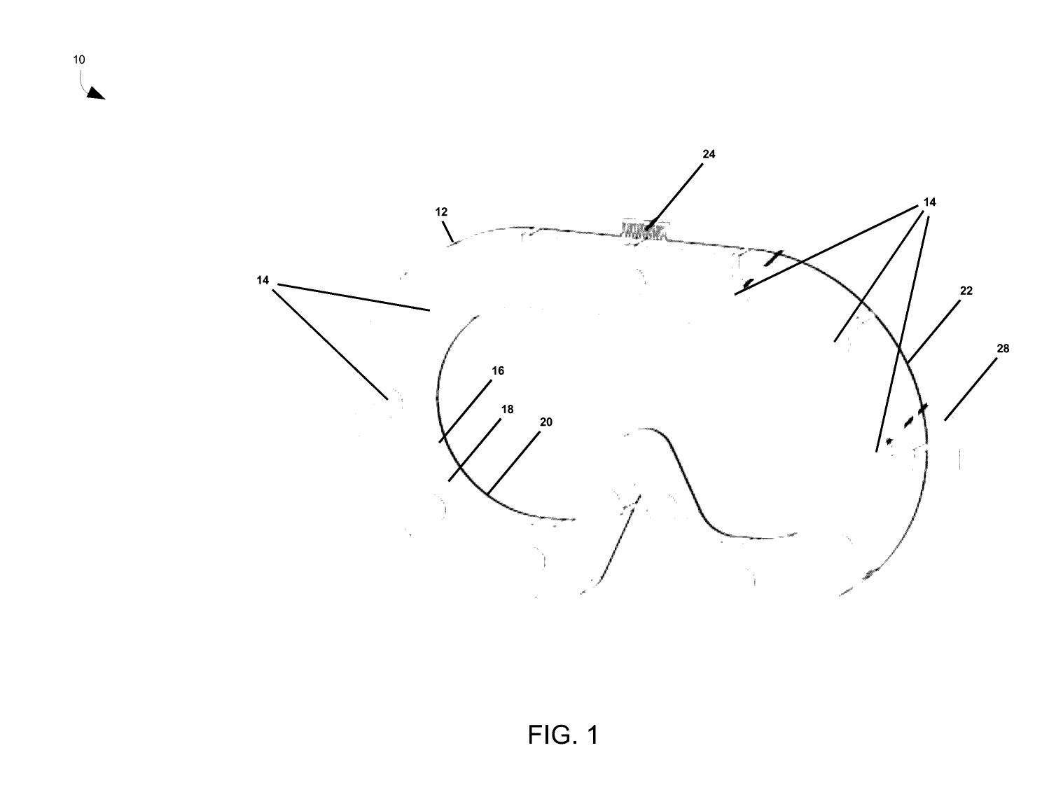

Below are exemplary implementations provided to enable persons skilled in the art to make or use the embodiments of the disclosure and are not intended to limit the scope of the disclosure. Reference will be made below in detail to exemplary embodiments of the invention, examples of which are illustrated in the accompanying drawings. Wherever possible, the same reference characters used throughout the drawings refer to the same or like parts, without duplicative description. For purposes of description herein, the terms “upper”, “lower”, “left”, “rear”, “right”, “front”, “vertical”, “horizontal”, and derivatives thereof, shall relate to the invention as oriented in FIG. 1. There is no intention to be bound by any expressed or implied theory presented in the preceding technical field, background, brief summary or the following detailed description. Specific dimensions and other physical characteristics relating to the embodiments disclosed herein are not to be considered as limiting, unless the claims expressly state otherwise.

Furthermore, as used herein, the terms “substantially,” “generally,” and “about” indicate conditions within reasonably achievable manufacturing and assembly tolerances, relative to ideal desired conditions suitable for achieving the functional purpose of a component or assembly. The term “real-time,” as used herein, means a level of processing responsiveness that a user senses as sufficiently immediate or that enables the processor to keep up with an external process. As used herein, “electrically coupled”, “electrically connected”, “electrical communication”, and “communication” mean that the referenced elements are directly or indirectly connected such that an electrical current, or other communication medium, may flow from one to the other. The connection may include a direct conductive connection, i.e., without an intervening capacitive, inductive or active element, an inductive connection, a capacitive connection, and/or any other suitable electrical connection. Intervening components may be present.

As is to be understood, the term “control system”, as used herein, is a device or set of devices that manage, govern or regulate the performance of another devise or system. Accordingly, an “open-loop control system”, as used herein, means a control system that receives an input signal, and based on that signal, produces a specific command or output without the need for feedback from the output device. In other words, an open-loop control system is controlled by the input signal irrespective of feedback in that the output command is simply dependent on the input signal such that any corrective action requires an operator input to change the behavior of the system to achieve a desired output condition. On the other hand, a “closed-loop control system”, as used herein, means a control system where the output produced by a command signal is measured, and consideration is given to the produced result for correction purposes, e.g., a thermostat used to control the temperature of an air-conditioning system where, once the desired temperature is reached, an input signal is sent to the controller which then sends a signal to turn off the air-conditioner. Further, it should be noted that “Electromyography (EMG)”, as used herein, refers to an electrodiagnostic medicine technique used for evaluating and recording the electrical activity produced by skeletal muscles.

Accordingly, exemplary embodiments of the present invention provide a device and method for EMG based facial gesture recognition as a means to deliver commands to a computer or other device. In circumstances where hands-free control of devices is desired, options for users are often limited to voice recognition, eye gaze tracking, and motion tracking of the head or limbs. The present invention allows an individual to control electronic devices, such as a VR system, by making brief facial gestures. Embodiments of the present invention uses electrode sensors positioned on the surface of the foam rim of a pair of goggles which are then pressed against a user's face when worn so as to capture the EMG signals of facial patterns. A means of programming and remembering pre-established patterns is also incorporated.

As will be understood, embodiments of the present invention are designed to capture EMG patterns of entire facial gestures, e.g., a compilation of EMG from many of the facial muscles, which activate during a given facial gesture, as opposed to EMG from single/individual muscles generated at specific facial muscle locations. In some embodiments, the user programs, configures or calibrates the device through a specific sequence so as to prepare the system/facial gesture controller to identify each of several facial gestures. Thus, when the facial gesture is made again it will be recognized by the system/facial gesture controller.

Referring now to FIG. 1, a facial gesture controller 10 for controlling an electronic device is shown in accordance with an embodiment of the present invention. The facial gesture controller 10 may include a body 12 and a plurality of EMG electrodes/sensors 14 disposed thereon. The body 12 may be configured to fit over the face of a user, and may include a foam pad 16 having a first side 18 and a second side 20. The foam pad 16 may be made of common industry materials that are typically used to provide comfort, e.g., materials possessing the ability to bend along facial contours, such as comfort foam and other non-foam soft padding materials.

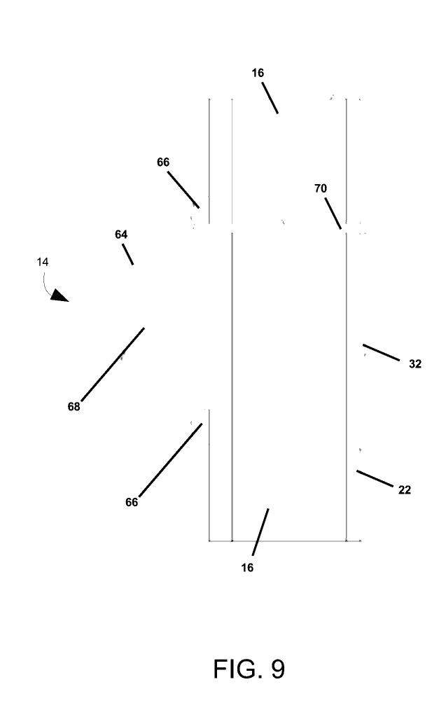

As shown in FIG. 1, the plurality of electrodes 14 may be disposed symmetrically around the circumference/perimeter of the body 12. As will be appreciated, however, the number, locations, shapes and sizes of the electrodes 14 may vary depending on the user and the desired application. The electrodes 14 may be metal, fabric, plastic, and/or any other conductive material that provides a comfortable taught fitting/contact with the skin. In embodiments the electrodes/sensors 14 are composed of silver fabric adhered to the foam surface 18, and may be applied directly onto the foam surface 18, or may be mounted on a slightly raised compressible features, or “bumps” (shown in FIG. 9), to further improve contact relative to the surrounding foam 16. For example, in embodiments, the electrodes 14 may be on an assembly (not shown) that is rolled over the foam pad 16 such that the electrodes adhere to the foam 16 to form a unitary device.

A ground and/or reference contact (not shown) may be included as an additional sensor contact on the foam 16, or may be removeably attached via a clip-to-an-earlobe or in any other standard manner that ground leads are held in contact with the skin during EMG measurements. The electrodes 14 may be wired as unipolar electrodes, differential electrodes, or in a combination of arrangements thereof to optimize signal to noise characteristics. The electrodes 14 may be arrayed along the length of the body 12 in any number or location. For example, some embodiments may utilize 4-12 electrodes of unipolar or differential configuration spread along the length of the body 12, e.g., so as to be aligned across the forehead, down the side of the face, under the eyes, and/or over the nose of a user. Alternative arrangements are possible depending on the shape and path of the body 12, which may change depending on the particular application the controller 10 is being used for, e.g., VR systems, sports, etc. In some embodiments, a user may be able to slide or adjust the electrode 14 positions along the body 12 to optimize EMG signal acquisition.

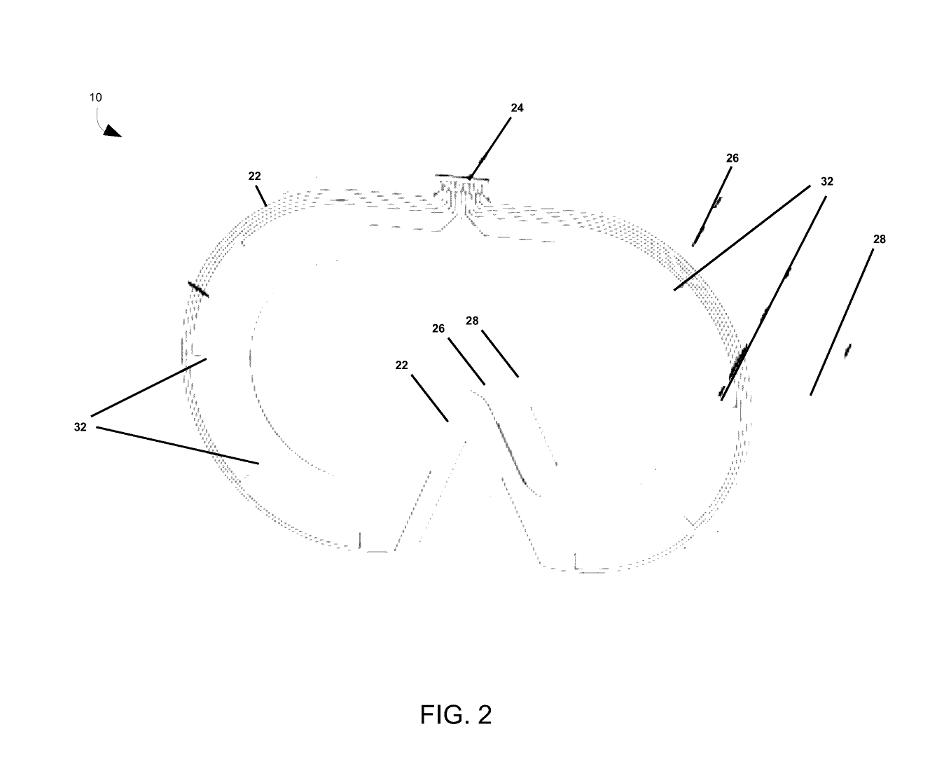

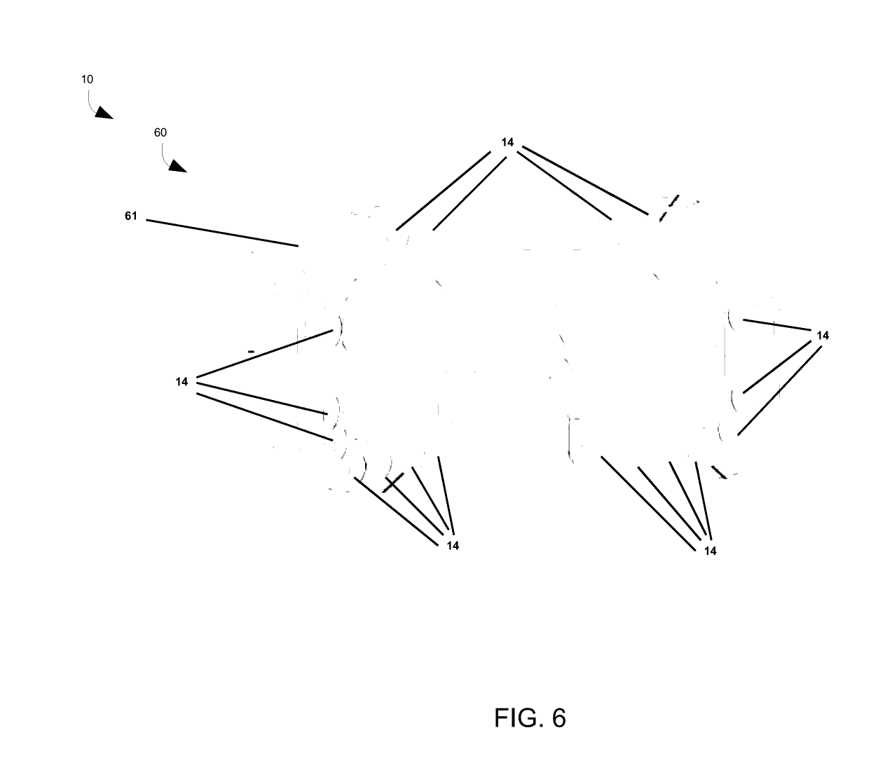

As illustrated in FIG. 2, the body 12 may further include a flexible printed circuit board (“PCB”) backing/conductive material layer 22 having a printed circuit board interface connector 24 which may be used to transfer the EMG and/or electrical signals to an interface device (not shown) as determined by the required functionality of the controller 10 and the device which is to receive command signals therefrom. In embodiments, the PCB 22 may be an Atmel or Chipkit: Open BCI 32 bit board V3-32, and/or another appropriate board that employs copper or other conductive materials. The body 12 may further include an adhesive layer 26 and an adhesive backing 28, which may provide for the PCB 22 to adhere to a frame of a pair of goggles (FIG. 6) or other structure. As is to be appreciated, in embodiments, hook and loop fasteners, and/or other methods of selectively attaching/fixing the body 12 to goggles or other structures may be utilized in place of, or in conjunction with, the adhesive layer 26 and/or backing 28.

Further, in some embodiments, the PCB 22 may utilize an additional adhesive layer that connects the PCB 22 to the soft foam pad 16 (FIG. 1). As will be appreciated, in embodiments, the additional adhesive layer may be incorporated into the comfort foam 16, e.g., an adhesive siding attached to one of the surfaces 18, 20 and having a removable paper backing, which is removed prior to fixing the body 12 to the PCB 22.

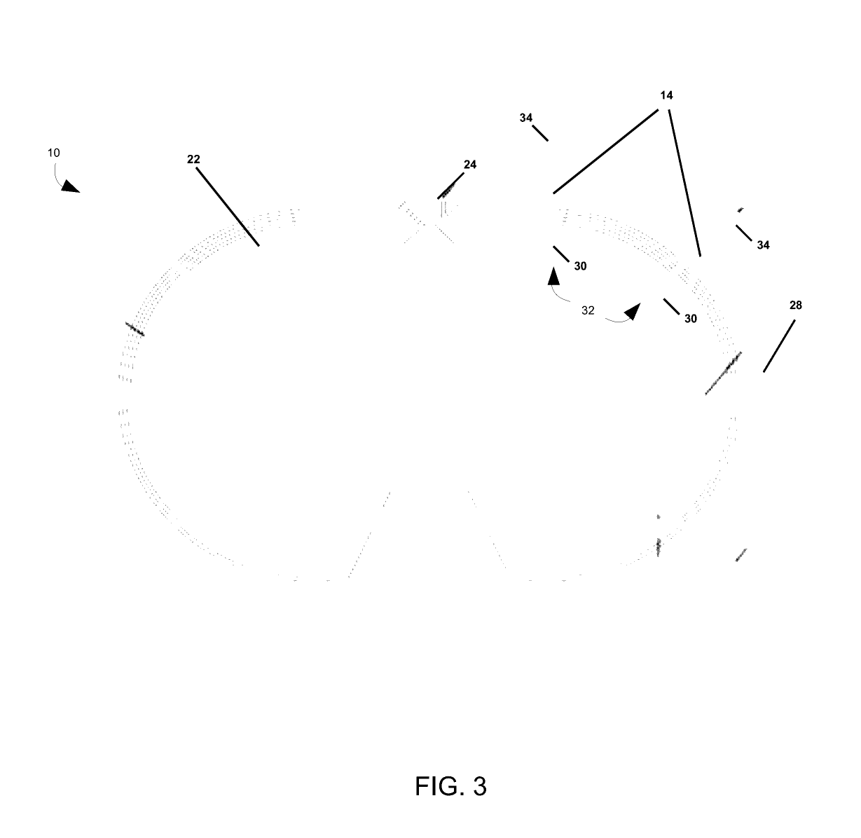

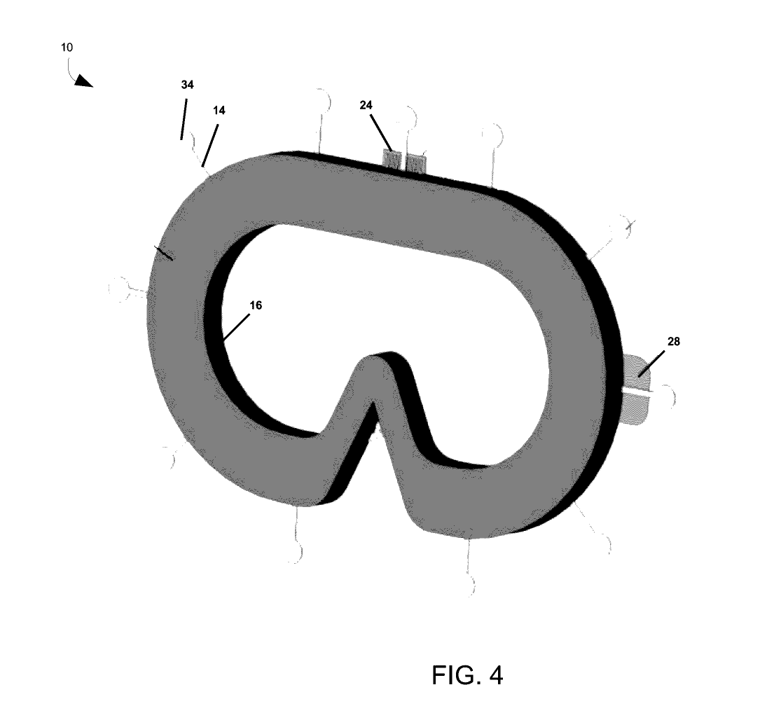

Turning now to FIG. 3, the electrodes 14 may be silver cloth electrodes having a first end 30 connected to connection sites/electrode contacts 32, the positions of which correspond to the arrangement of the electrodes 14, on the PCB 22 with a second end 34 free to wrap around the foam pad 16 (FIG. 1) so as to adhere to the surface of the first side 18 (FIG. 1) of the foam pad 16 such that the electrodes 14 contact the skin of a user over the facial muscles. In other words, the sensors 14 may be folded from an extended position (shown in FIGS. 3 and 4), around an adjacent peripheral area of the body 12/soft pad 16 to a folded position (shown in FIG. 1) while remaining electronically connected to the conductive material layer/PCB 22. In such embodiments, the sensors 14 may be in taut contact with the skin of the face of the user when in the folded position.

As is to be understood, the first end 30 of the silver cloth electrodes 14 may be attached to the PCB 22 using any suitable conductive adhesive, and permit transmission of electrical signals in response to the movements of muscles underneath the skin of the user. The sensors 14 may be configured to pick up/detect the slightest signals, e.g., on the order of millivolts, coming from the muscles underneath skin of the user. The sensors may form a footprint, which is fashioned, in the form of an array around a particular area of the face.

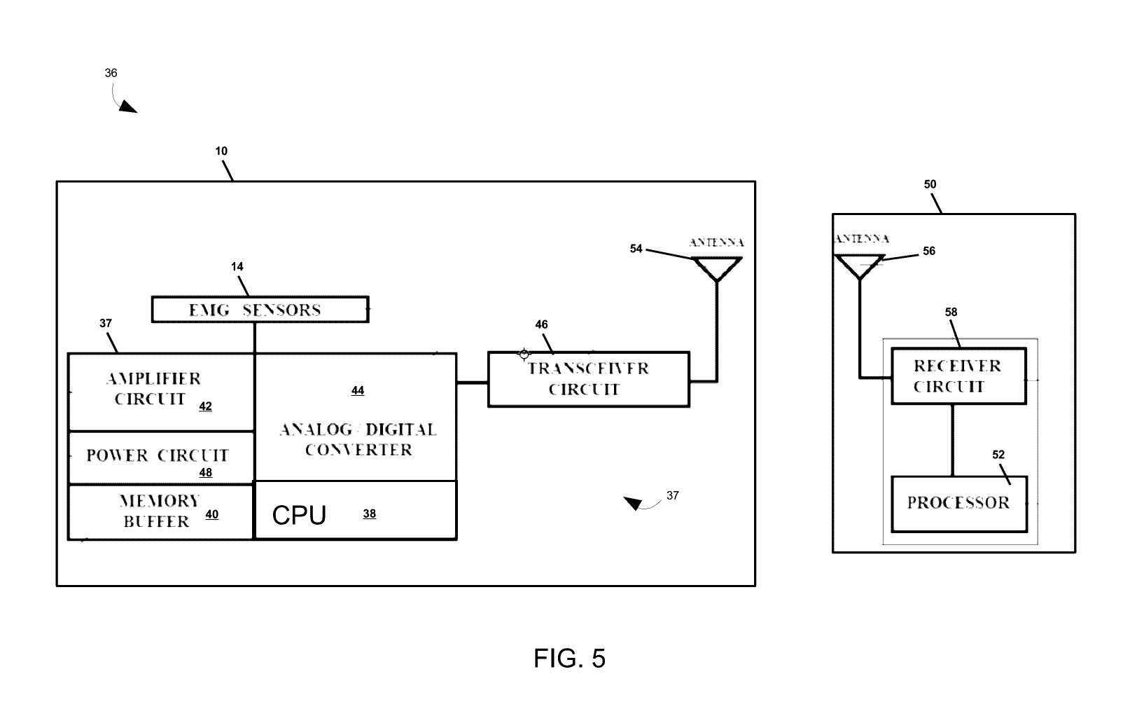

Referring now to FIG. 5, a block diagram of a system 36 that incorporates an embodiment of the facial gesture controller 10 is shown. The controller 10 and/or system 36 may include an electronics module 37 to which the sensors 14 may connect via embedded wires and/or via the connector 24. Such wires may run directly to the electronics module 37, or can be incorporated into the PCB 22 mounted behind the foam 16 or elsewhere on the body 12. As is to be understood, the electronics module 37 performs basic signal processing and outputting of processed/control signals to a computer or other target device. Accordingly, the electronics module 37 may include a suitable processor/CPU 38 (which may be embedded in or on the body 12), a software application maintained/stored in a memory buffer 40 which may also store the EMG signals prior to processing, an amplifier circuit 42 that amplifies/boosts detected EMG signals, an analogue to digital (“a/d”) converter 44 that converts the EMG/electronic signals into digital form, and a transceiver circuit 46, which are all powered by a power circuit 48 which may be controlled by a power switch (not shown) that may be toggled manually and/or automatically. In embodiments, the controller 10 and/or electronics module 37 may be powered with a rechargeable battery or via direct wired connection to another electronic device apart from the controller 10. The transceiver 46 may transmit the control signals, which are then wirelessly received by a corresponding electrical item/device 50, e.g., a smart phone, computer, VR system, or other home electronic, etc. As further shown in FIG. 5, the device 50 may include a programmed processor 52.

As is to be further appreciated, the system 36 may further include a sending antenna 54 in electronic communication with the transceiver circuit 46 and configured for Wi-Fi, blue tooth, or other suitable wireless communication protocols. The system 36 may further include a receiving antenna 56, disposed within the device 50, that picks up/receives the transmitted control signals signal via receiver circuit 58, also disposed within the device 50. The received control signals may then be forwarded to the processor 52 where further processing may take place.

Additionally, it is to be understood that embodiments of the present invention may use and/or be integrated with USB power sources, various controllers (including voltage controllers), back up battery systems, power boards, amplifiers, analog to digital converters, CPUs, Bluetooth devices, computers, and/or phone connections. Thus, as can be seen, the facial gesture controller 10 may be a standalone device, or integrated/attached/in electronic communication with another electronic device 50. Additionally, while the embodiments herein depict the processing/conversion of EMG signals into control signals as occurring within the CPU 38 disposed on the controller 10, it is to be understood that in other embodiments, the digital processing/conversion of the EMG signals into control signals may be performed by the processor 52 of the electronic device, or other suitable processer located apart from the facial gesture controller 10. For example, in embodiments, the EMG signals may be transmitted over a wired connection to a computer running a VR system.

Thus, in operation according to an embodiment, the facial gesture controller 10 measures bio signals of a user generated by the movements of one or more facial muscles via the sensors 14, which in response generate and transmit electrical signals to the CPU 38. The CPU 38 then generates/converts the received electrical signals into control signals, and then transmits the control signals to the electronic device 50.

As stated above, in embodiments, the facial gestures may correspond to one or more commands. In such embodiments, the following four basic default gestures may be detected/utilized by the facial gesture controller: 1) raised eyebrows; 2) a wrinkled nose; 3) a left smirk; and 4) a right smirk. As is to be understood, however, the facial gesture controller 10 may further learn additional facial gestures, and/or a user's custom facial control gestures, learned by the controller 10 through the use of the software application stored in memory device 40 when loaded into CPU 38. For example, in embodiments, the user may train the facial gesture controller 10, through a brief calibration sequence, to recognize uncommon facial gestures, which are not normally produced through regular activity, and to associate such gestures with computer commands, e.g., a mouse click. As will be appreciated, uncommon facial gestures may be chosen by the user such that they are easy to make, yet not easily made by accident or over the course of a normal day.

In some embodiments, the user may calibrate the facial gesture controller 10 by attempting to associate a particular control instruction with several candidate facial gestures, with the facial gesture controller 10 suggesting/paring/selecting the candidate gesture most easily differentiated from other EMG “noise” (such as speaking, head motion, or other common facial gestures). The facial gesture controller 10 may then recognize/associate the selected facial gesture as a command to “activate” or modulate functions controlled by an electronic device. For example, a user may raise one or both eyebrows or wrinkle their nose to “click” on something in “virtual space,” which may correspond to a traditional “button press” during game play or other applications. As another example, the facial gesture controller 10 may capture EMG from many facial muscles, and generate a “click” control signal only if certain muscles are actively producing EMG while other muscles are simultaneously silent of EMG. As will be appreciated, exemplary EMG patterns used to trigger control signals may be unique to a given facial gesture so as to make it unlikely to be produced by accident while talking, moving, or performing other common tasks.

Thus, as shown above, by recognizing specific preprogrammed idiosyncratic facial gestures, embodiments of the present invention are able to associate idiosyncratic facial gestures to control functions, which in turn, provides for a user to control an electronic device 50 via distinct/customized facial gestures.

Continuing on, some embodiments of the facial gesture controller 10 may detect the emotions of the person using it, and in response send an emotion-specific command. For example, in addition to being able to identify idiosyncratic gestures that represent a “button press”, embodiments of the present invention may also learn to recognize facial gestures that correspond to a user's emotional state, so that such emotions can be interpreted by the facial gesture controller 10 and represented by the electronic device 50. For example, the facial gesture controller 10 may provide for a virtual avatar to express the emotional state of its user to others in a virtual world by representing that emotional state of the user on the face of the user's avatar. Thus, as can be appreciated, embodiments of the facial gesture controller 10 allow users to communicate feelings in a passive and natural state, rather than requiring users to actively select and update their emotional state, via a keyboard or other hand-based device, thereby making communication more natural and the corresponding information richer than would otherwise be possible.

Referring now to FIG. 6, a point of innovation lies in the ability of the padded foam 16 to be employed as liner for a pair of goggles 60 while having the sensors 14 embedded within. Thus, as is to be appreciated, the body 12 may be incorporated into, or otherwise form part of, a goggle frame 61. By incorporating the sensors 14 into a goggle frame 61, embodiments of the present invention provide for the sensors 14 to be automatically positioned correctly over the appropriate facial muscles, thereby allowing the wearer to control target devices with distinct facial gestures in a silent and hands-free manner. As is to be understood, the goggles 60 and/or frame 61 can be of any nature, e.g., for virtual reality, sports, or any other activities requiring goggles. It is to be understood that the goggles 60 do not have to be of a particular type, and in embodiments, may even be placed over the skin without a rigid frame 61.

As shown in FIG. 6, the body 12/foam 16 may be permanently or removeably attached to the inner surface of a goggle frame 61 with the EMG sensors 14 disposed so as to fit around the face of a user. Accordingly, the electrode sensors 14 may be embedded into or onto the foam perimeter of the goggles 60 so as to contact the skin around the face. The sensors 14, however, may also be incorporated into rubber, plastic or metal layers that may be used instead of foam in some types of goggles.

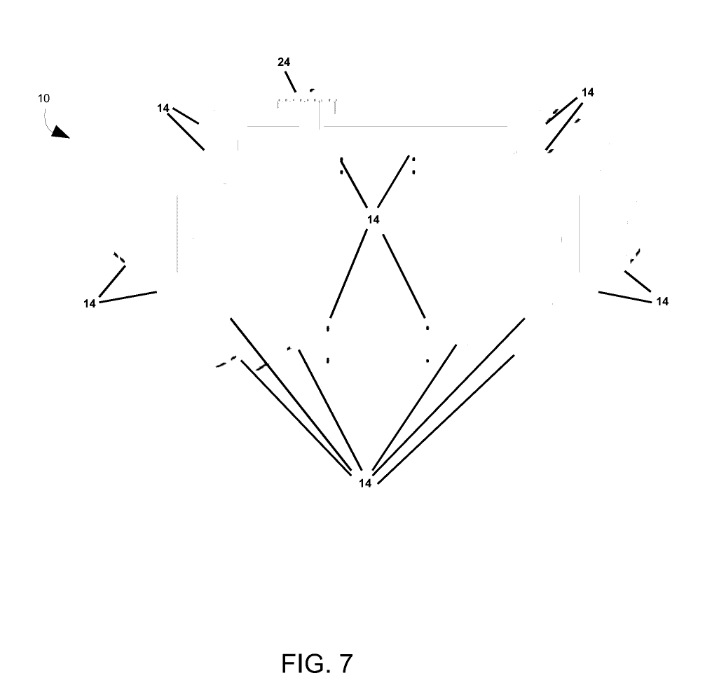

While FIG. 6 depicts a sensor electrode 14 array configuration appropriate for use in VR environments, wherein the array is positioned about the eyes while remaining open in the center, is to be understood, that other embodiments, e.g., the array depicted in FIG. 7, should not be construed as outside the scope of the invention.

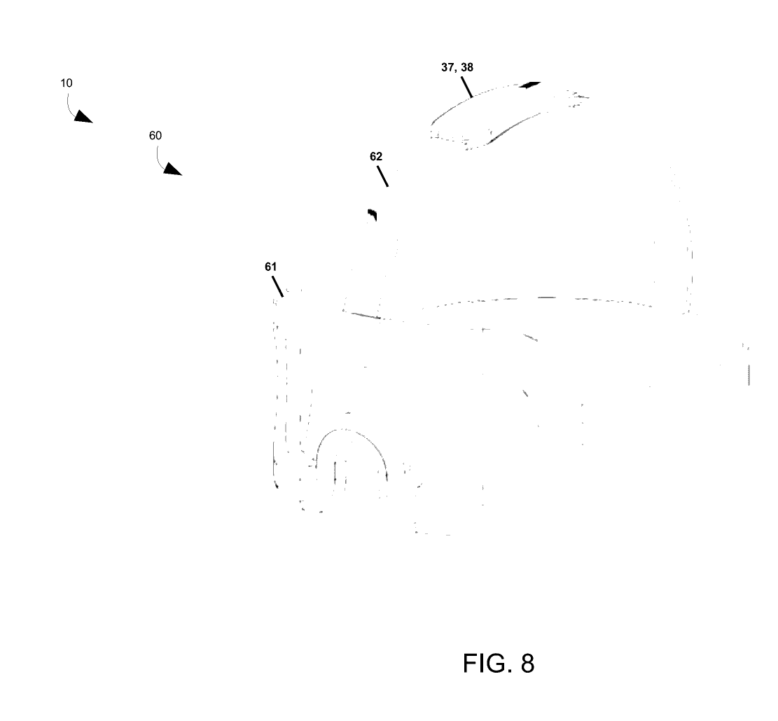

Further, as shown in FIG. 8, in embodiments where the controller 10 forms part of a pair of goggles 60, the electronics module 37 and/or CPU 38 may be separated/apart from the sensor 14 array by being carried/disposed/mounted on a strap 62 connected to the facial gesture controller 10 and/or goggles 60.

Turning now to FIG. 9, a cross section of an embodiment of a sensor/electrode 14 having a raised form is shown. As can be seen, the electrode 14 may be made of conductive material 64 stitched circumferentially using stitches 66 in a manner that raise the electrode 14 above a base material layer 16. In embodiments, the conductive material 64 may increase the contact area and conductivity between the electrode 14 and the skin of the user. Additionally, the conductive material 64 may be a cloth like material, e.g., silver, that coats the electrode 14 so as to mitigate wear and tear resulting from repeated use of the facial gesture controller 10. In such embodiments, the silver conductive material 64 may be made to rise and have a snug fit against the skin of the user by using embroidery material 68 underneath the conductive material 64, thereby giving the electrode 14 a protruding/raised configuration. Thus, when an individual puts on/wears the goggles, the silver fabric is pressed between the foam backing 16 and the skin of the face around the eyes. As is to be appreciated, silver has excellent ion conductivity, is bio compatible, e.g., causes very little allergic reaction, and does not leave green corrosion spots.

In embodiments, the stitches 66 may be made of polyester stitch material, silk, Rayon, and/or another appropriate material capable of stitching around the center silver embroidered conductive fabric 64. As is to be appreciated, Rayon or silk stitching provides a circumference for the conductive electrode 14 to stand out around the embroidery material 68. Further, in embodiments, the conductive electrode 14 may be constructed of a silver impregnated fabric, which provides excellent conductive qualities. Similarly, other appropriate conductive or conductively coated fabrics may include stainless steel, nickel, copper, and polyester.

The silver coated electrodes 14 may be attached to the PCB 22 using a conductive adhesive. Additionally, in embodiments, the cloth layer 68 may also provide for the electrode 14 to be sewn on to it while also acting as a cover for the base material layer 16, e.g., the soft foam base. Further, in embodiments, a stainless steel thread or wire 70 may be threaded through the silver material layer 64, cloth layer 68, soft foam material base layer 16, and flexible PCB 22 to a copper electrode surface 32 of the PCB layer 22. In such embodiments, the thin stainless steel thread 70 acts a conductor and provides a clear path for an EMG signal to travel from the silver electrode 14 to the PCB 22.

Thus, some embodiments provide for the conductive sensors to be sewn to the surface of the foam and connected to the flexible PCB backing with a wire or conductive thread, that travels/passes through the foam, so as to provide distinct locations were the EMG sensors can obtain desired signals.

It is also to be understood that the system 36 and/or facial gesture controller 10 may include the necessary electronics, software, memory, storage, databases, firmware, logic/state machines, microprocessors, communication links, displays or other visual or audio user interfaces, printing devices, and any other input/output interfaces to perform the functions described herein and/or to achieve the results described herein. For example, as previously mentioned, the facial gesture controller 10 may include at least one processor 38, and system memory/data storage structures 40, which may include random access memory (RAM) and read-only memory (ROM). The at least one processor 38 may include one or more conventional microprocessors and one or more supplementary co-processors such as math co-processors or the like. The data storage structures discussed herein may include an appropriate combination of magnetic, optical and/or semiconductor memory, and may include, for example, RAM, ROM, flash drive, an optical disc such as a compact disc and/or a hard disk or drive.

Additionally, the software application may be read into a main memory of the at least one processor 38 from a computer-readable medium. The term “computer-readable medium”, as used herein, refers to any medium that provides or participates in providing instructions to the at least one processor 38 (or any other processor of a device described herein) for execution. Such a medium may take many forms, including but not limited to, non-volatile media and volatile media. Non-volatile media include, for example, optical, magnetic, or opto-magnetic disks, such as memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, a RAM, a PROM, an EPROM or EEPROM (electronically erasable programmable read-only memory), a FLASH-EEPROM, any other memory chip or cartridge, or any other medium from which a computer can read.

While in embodiments, the execution of sequences of instructions in the software application causes the at least one processor to perform the methods/processes described herein, hard-wired circuitry may be used in place of, or in combination with, software instructions for implementation of the methods/processes of the present invention. Therefore, embodiments of the present invention are not limited to any specific combination of hardware and/or software.

It is further to be understood that the above description is intended to be illustrative, and not restrictive. For example, the above-described embodiments (and/or aspects thereof) may be used in combination with each other. Additionally, many modifications may be made to adapt a particular situation or material to the teachings of the invention without departing from its scope.

For example, in an embodiment, a facial gesture controller for controlling an electronic device is provided. The facial gesture controller includes a body, a plurality of sensors, and at least one processor. The body is configured to fit over the face of a user. The plurality of sensors are disposed on the body. The at least one processor is in electrical communication with the sensors. The sensors generate and transmit electrical signals to the at least one processor in response to movements of one or more facial muscles of the user. The at least one processor generates and transmits control signals corresponding to the electrical signals, received from the sensors, to the electronic device. In certain embodiments, the body is a soft pad having a first side opposite a second side. In certain embodiments, the facial gesture controller further includes a conductive material layer disposed on the first side, and the sensors are mounted on the second side. In certain embodiments, the sensors fold around an adjacent peripheral area of the soft pad and are electronically connected to the conductive material layer such that the sensors transmit the electrical signals to the at least one processor via the conductive material layer. In certain embodiments, the movements of the one or more facial muscles of the user correspond to at least one of a raising of an eyebrow, a wrinkling of a nose, and a smirk. In certain embodiments, the smirk is a left smirk. In certain embodiment, the smirk is a right smirk. In certain embodiments, the body forms part of a pair goggles. In certain embodiments, the sensors extend outward from the body and are in taut contact with the skin of the face of the user. In certain embodiments, the sensors generate the electrical signals in response to sensing millivolt signals generated by the movements of the one or more facial muscles of the user. In certain embodiments, the sensors include a conductive material layer sewn to a cloth layer. In certain embodiments, the facial gesture controller further includes a conductive thread disposed within the body so as to form a conductive conduit between a first side of the body and a second side of the body.

Other embodiments provide for a computerized system. The system includes an electronic device, and a facial gesture controller. The facial gesture controller includes at least one processor, a body configured to fit over the face of a user, and a plurality of sensors disposed on the body. The sensors generate and transmit electrical signals to the at least one processor in response to movements of one or more facial muscles of the user. The at least one processor generates and transmits control signals corresponding to the electrical signals, received from the sensors, to the electronic device. In certain embodiments, the body is a soft pad having a first side opposite a second side, the facial gesture controller further includes a conductive material layer disposed on the first side, and the sensors are mounted on the second side. In certain embodiments, the movements of the one or more facial muscles of the user correspond to at least one of a raising of an eyebrow, a wrinkling of a nose, and a smirk. In certain embodiments, the body forms part of a pair of goggles. In certain embodiments, the sensors generate the electrical signals in response to sensing millivolt signals generated by the movements of the one or more facial muscles of the user.

Yet still other embodiments provide for a method of controlling an electronic device. The method includes: generating electrical signals in response to movements of one or more facial muscles of a user via a plurality of sensors disposed on a body of a facial gesture controller, the body configured to fit over the face of the user; transmitting the generated electrical signals to at least one processor; generating control signals based at least in part on the electrical signals via the at least one processor; and transmitting the control signals to the electronic device via the at least one processor. In certain embodiments, generating electrical signals in response to movements of one or more facial muscles of a user via a plurality of sensors disposed on a body of a facial gesture controller, the body configured to fit over the face of the user includes: detecting, via the sensors, at least one of a raising of an eyebrow, a wrinkling of a nose, and a smirk. In certain embodiments, generating electrical signals in response to movements of one or more facial muscles of a user via a plurality of sensors disposed on a body of a facial gesture controller, the body configured to fit over the face of the user includes: detecting millivolt signals generated by the movements of the one or more facial muscles via the sensors.

In yet still other embodiments, the facial gesture controller may include several layers of flexible PCB with a front layer made of comfort foam, wherein a connector is used to connect to a generic electronics module.

In yet still other embodiments, a comfort foam with an adhesive backing is applied to the flex circuit/PCB and silver electrodes are rolled over the foam and adhered to the conductive material layer using a conductive adhesive. The electrodes wrap around the foam material completing the circuit. Conductive square pads may be placed underneath sensors for transference of the EMG signals. The number of electrodes used may vary depending on the intended application and desired functionality the controller. The sensors may be unipolar, meaning that only one sensor input is required, or differential, meaning a pair of electrodes are used to measure the signal, or a combination thereof so as to optimize noise characteristics.

In yet still further embodiments, the electrodes may be configured in different ways singularly or differentially, meaning in pairs. In such embodiments, distinct facial gestures produce a pattern of distinct EMG signals at predetermined electrode locations which are then converted to a command signals. Thus, some embodiments of the present invention are similar in nature to an open-loop system, in that input in the form of EMG signals is received and used to produce an output command signal without the need for feedback.

Accordingly, by focusing on facial EMG pattern recognition, via a body having sensors incorporated/embedded therein, some embodiments of the invention provide for the transmission of control signals to an electronic device resulting from detected facial gestures, thereby providing for the exchange of non-verbal communications over electronic forms of communication.

Additionally, by providing for extreme flexibility, via the foam pad 16 and PCB 22, some embodiments of the facial gesture controller may be used in low profile devices. In other words, the foam layer with the embedded sensors may be very adaptable in use.

Further, some embodiments may provide for a universal controller for VR systems, or another system where hands free control is desirable, having clear benefits over existing controller technology in that embodiments of the present invention remove the need for a hand controller, thereby alleviating the urge to remove the VR het set and avoiding the associated disorientation.

Moreover, in embodiments where the electrodes are incorporated into a preexisting foam layer of goggles, no additional equipment or surfaces need be worn by the user. Thus, the controller 10 may be seamlessly integrated into VR goggles without any change in their appearance, their feel, or their comfort.

Further still, because facial gestures are usually silent, quickly produced, and/or easily learned, embodiments of the present invention provide for an ideal means of command signaling for many individuals.

While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format and are not intended to be interpreted based on 35 U.S.C. §112(f), unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A facial gesture controller for controlling an electronic device, the facial gesture controller comprising:
a body configured to fit over the face of a user;
a plurality of sensors disposed on the body;
at least one processor in electrical communication with the sensors; and
wherein the sensors generate and transmit electrical signals to the at least one processor in response to movements of one or more facial muscles of the user, and
the at least one processor generates and transmits control signals corresponding to the electrical signals, received from the sensors, to the electronic device.

2. The facial gesture controller of claim 1, wherein the body is a soft pad having a first side opposite a second side.

3. The facial gesture controller of claim 2 further comprising a conductive material layer disposed on the first side; and
wherein the sensors are mounted on the second side.

4. The facial gesture controller of claim 3, wherein the sensors fold around an adjacent peripheral area of the soft pad and are electronically connected to the conductive material layer such that the sensors transmit the electrical signals to the at least one processor via the conductive material layer.

5. The facial gesture controller of claim 1, wherein the movements of the one or more facial muscles of the user correspond to at least one of a raising of an eyebrow, a wrinkling of a nose, and a smirk.

6. The facial gesture controller of claim 5, wherein the smirk is a left smirk.

7. The facial gesture controller of claim 5, wherein the smirk is a right smirk.

8. The facial gesture controller of claim 1, wherein the body forms part of a pair goggles.

9. The facial gesture controller of claim 1, wherein the sensors extend outward from the body and are in taut contact with the skin of the face of the user.

10. The facial gesture controller of claim 1, wherein the sensors generate the electrical signals in response to sensing millivolt signals generated by the movements of the one or more facial muscles of the user.

11. The facial gesture controller of claim 1, wherein the sensors comprise a conductive material layer sewn to a cloth layer.

12. The facial gesture controller of claim 1 further comprising:
a conductive thread disposed within the body so as to form a conductive conduit between a first side of the body and a second side of the body.

13. A computerized system comprising:
an electronic device; and
a facial gesture controller that includes:
at least one processor;
a body configured to fit over the face of a user; and
a plurality of sensors disposed on the body; and
wherein the sensors generate and transmit electrical signals to the at least one processor in response to movements of one or more facial muscles of the user, and
the at least one processor generates and transmits control signals corresponding to the electrical signals, received from the sensors, to the electronic device.

14. The computerized system of claim 13, wherein
the body is a soft pad having a first side opposite a second side,
the facial gesture controller further includes a conductive material layer disposed on the first side, and
the sensors are mounted on the second side.

15. The computerized system of claim 13, wherein the movements of the one or more facial muscles of the user correspond to at least one of a raising of an eyebrow, a wrinkling of a nose, and a smirk.

16. The computerized system of claim 13, wherein the body forms part of a pair of goggles.

17. The computerized system of claim 13, wherein the sensors generate the electrical signals in response to sensing millivolt signals generated by the movements of the one or more facial muscles of the user.

18. A method of controlling an electronic device, the method comprising:
generating electrical signals in response to movements of one or more facial muscles of a user via a plurality of sensors disposed on a body of a facial gesture controller, the body configured to fit over the face of the user;
transmitting the generated electrical signals to at least one processor;
generating control signals based at least in part on the electrical signals via the at least one processor; and
transmitting the control signals to the electronic device via the at least one processor.

19. The method of claim 18, wherein generating electrical signals in response to movements of one or more facial muscles of a user via a plurality of sensors disposed on a body of a facial gesture controller, the body configured to fit over the face of the user comprises:
detecting, via the sensors, at least one of a raising of an eyebrow, a wrinkling of a nose, and a smirk.

20. The method of claim 18, wherein generating electrical signals in response to movements of one or more facial muscles of a user via a plurality of sensors disposed on a body of a facial gesture controller, the body configured to fit over the face of the user comprises:
detecting millivolt signals generated by the movements of the one or more facial muscles via the sensors.
